(19) United States
(12) Patent Application Publication
(10) Pub. No.: US 2014/0005558 A1
(43) Pub. Date: Jan. 2, 2014

(54) PRESSURE SENSING GUIDEWIRE

(71) Applicant: BOSTON SCIENTIFIC SCIMED, INC., MAPLE GROVE, MN (US)

(72) Inventor: DANIEL J. GREGORICH, ST. LOUIS PARK, MN (US)

(21) Appl. No.: 13/931,324

(22) Filed: Jun. 28, 2013

Related U.S. Application Data
(60) Provisional application No. 61/666,245, filed on Jun. 29, 2012.

(51) Int. Cl. A61B 5/0215; A61B 5/00

(52) U.S. Cl. CPC A61B 5/02154; A61B 5/6851; A61B 5/0084
USPC 600/480; 600/486

(57) ABSTRACT

Medical devices and methods for making and using medical devices are disclosed. An example medical device includes a pressure sensing guidewire. The pressure sensing guidewire may include a shaft having a proximal portion, a distal portion, and a distal tip portion. The distal portion may have a plurality of slots formed therein. A pressure sensor may be disposed within the distal portion of the shaft.

### CROSS-REFERENCE TO RELATED APPLICATIONS

This application claims priority under 35 U.S.C. §119 to U.S. Provisional Application Ser. No. 61/666,245, filed Jun. 29, 2012, the entirety of which is incorporated herein by reference.

### TECHNICAL FIELD

The present disclosure pertains to medical devices, and methods for manufacturing medical devices. More particularly, the present disclosure pertains to blood pressure sensing guidewires.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device includes a pressure sensing guidewire. The pressure sensing guidewire may include a shaft having a proximal portion, a distal portion, and a distal tip portion. The distal portion may have a plurality of slots formed therein. A pressure sensor may be disposed within the distal portion of the shaft.

Another example pressure sensing guidewire may include a tubular member having a proximal portion, a distal portion, and a lumen defined therein. The distal portion may have a plurality of slots formed therein. The plurality of slots may be configured to allow fluid to flow from an outer surface of the tubular member, through the slots, and into the lumen. A pressure sensor may be disposed within the lumen of the tubular member and may be positioned within the distal portion of the tubular member. A tip member may be coupled to the distal portion of the tubular member.

A pressure sensing guidewire system is also disclosed. The pressure sensing guidewire system may include a tubular member having a proximal portion, a slotted portion, and a lumen defined therein. The slotted portion may have a plurality of slots formed therein. The plurality of slots may be configured to allow fluid to flow from an outer surface of the tubular member, through the slots, and into the lumen. An optical pressure sensor may be disposed within the lumen of the tubular member and may be positioned within the distal portion of the tubular member. A fiber optic cable may be attached to the optical pressure sensor and may extend proximally therefrom. A handle member may be coupled to the proximal portion of the tubular member and the fiber optic cable. The system may also include an interferometer and a cable extending between the handle member and the interferometer.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional side view of a portion of an example medical device;

FIG. 2 is a side view of a portion of an example tubular member;

FIG. 3 is a side view of a portion of another example tubular member; and

FIG. 4 is a cross-sectional side view of a portion of another example medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

During some medical interventions, it may be desirable to measure and/or monitor the blood pressure within a blood vessel. For example, some medical devices may include pressure sensors that allow a clinician to monitor blood pressure. Such devices may be useful in determining fractional flow reserve (FFR), which may be understood as the pressure after a stenosis relative to the pressure before the stenosis. A number of pressure sensing devices, however, may pose technical challenges for steering, tracking, torqueing or otherwise navigating the device within the vasculature. For example, medical devices may include a relatively stiff pressure sensor located at or near the distal tip of the device and/or a sensor housing (in which the sensor is mounted) that may also be relatively stiff. Disclosed herein are a number of medical device that include pressure sensing capabilities and may be more easily steered, tracked, torqued, and/or otherwise navigated through the anatomy.

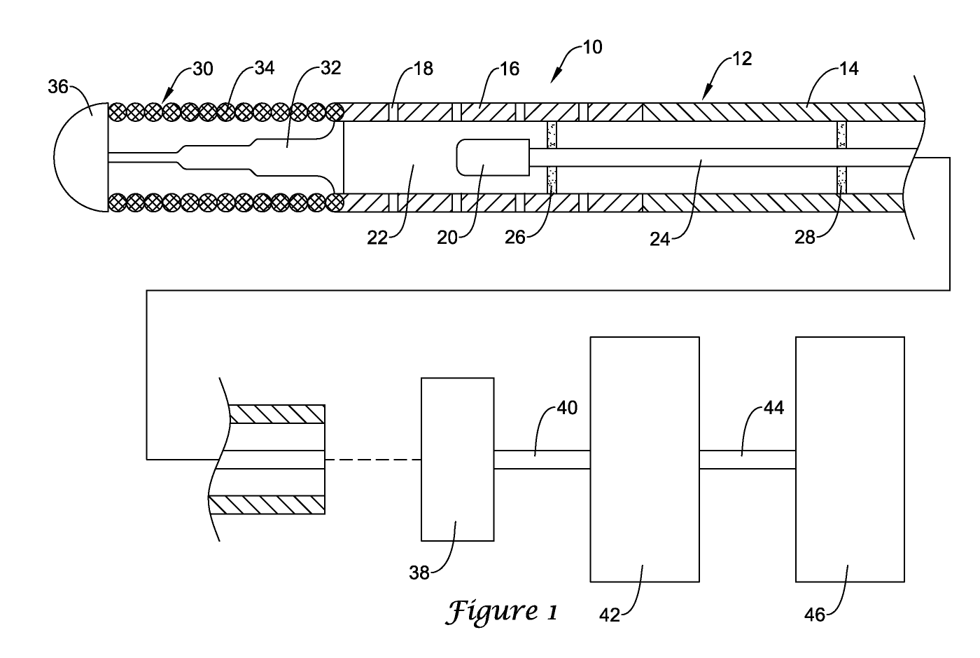

FIG. 1 illustrates a portion of an example medical device 10. In this example, medical device 10 is a blood pressure sensing guidewire 10. However, this is not intended to be limiting as other medical devices are contemplated including, for example, catheters, shafts, leads, wires, or the like. Guidewire 10 may include a guidewire shaft or tubular member 12. Tubular member 12 may include a proximal portion 14 and a distal portion 16. In some embodiments, proximal portion 14 and distal portion 16 are simply portions of the same monolith of material. In other embodiments, proximal portion 14 and distal portion 16 are discrete members that are attached to one another using a suitable attachment process (e.g., solder, weld, adhesive, or the like).

A plurality of slots 18 may be formed in tubular member 12. In at least some embodiments, slots 18 are formed in distal portion 16. In at least some embodiments, proximal portion 14 lacks slots 18. However, proximal portion 14 may include slots 18. Slots 18 may be desirable for a number of reasons. For example, slots 18 may provide a desirable level of flexibility to tubular member 12 (e.g., along distal portion 16) while also allowing suitable transmission of torque.

A pressure sensor 20 may be disposed within tubular member 12 (e.g., within a lumen 22 of tubular member 12). While pressure sensor 20 is shown schematically in FIG. 1, it can be appreciated that the structural form and/or type of pressure sensor 20 may vary. For example, pressure sensor 20 may include a semiconductor (e.g., silicon wafer) pressure senor, piezoelectric pressure sensor, a fiber optic or optical pressure sensor, a Fabry-Perot type pressure sensor, an ultrasound transducer and/or ultrasound pressure sensor, a magnetic pressure sensor, a solid-state pressure sensor, or the like, or any other suitable pressure sensor.

Typically, pressure sensors in guidewires are mounted within a mount or mounting structure at the distal end of the guidewire. The mount may take the form of a hypotube with a side hole or opening formed therein that provides access for the blood to reach the pressure sensor. Because the pressure sensor itself may be somewhat rigid and/or stiff and because the mount may also be somewhat rigid and/or stiff, such a configuration may define a region with increased stiffness at or near the distal end of the guidewire. This could pose technical challenges for navigating the guidewire within the vasculature.

The use of tubular member 12 (e.g., distal portion 16 having slots 18 formed therein) may improve the overall flexibility profile of guidewire 10 and/or improve the navigation, steerability, and trackability of guidewire 10. For example, the flexibility of distal portion 16 may be reduced when compared to a typical hypotube pressure sensor mount. Furthermore, the design of distal portion 16 can be tailored to provide a flexibility profile suitable for a given guidewire/intervention through a variety of different patterns and/or configurations for slots 18. Numerous slot configurations are contemplated including those disclosed herein.

Moreover, slots 18 may define a fluid pathway that allows blood (and/or a body fluid) to flow from a position along the exterior or outer surface of guidewire 10 (and/or tubular member 12), through slots 18, and into the lumen 22 of tubular member 12, where the blood can come into contact with pressure sensor 20. Because of this, no additional side openings/holes (e.g., other than slots 18) may be necessary in tubular member 12 for pressure measurement. This may also allow the length of distal portion 16 to be shorter than typical sensor mounts or hypotubes that would need to have a length sufficient for a suitable opening/hole (e.g., a suitable “large” opening/hole) to be formed therein that provides fluid access to sensor 20.

In use, a clinician may use guidewire 10 to measure or calculate FFR (e.g., the pressure after an intravascular lesion relative to the pressure before the lesion). This may include taking an initial pressure reading before or upstream of the lesion and then a comparative reading after or downstream of the lesion. This may also include monitoring the pressure while advancing guidewire 10 through a blood vessel until a pressure differential or drop in pressure is observed, indicating that guidewire 10 has reached and/or partially past the lesion as well as monitoring increases in pressure during and/or following a treatment intervention. In some embodiments, a second pressure measuring device may be used to measure pressure at another intravascular location and this pressure may be utilized in the calculation of FFR or otherwise used as part of the intervention.

As indicated above, pressure sensor 20 may include an optical pressure sensor. In at least some of these embodiments, a fiber optic cable 24 is attached to pressure sensor 20 and extends proximally therefrom. An attachment member 26 may attach fiber optic cable 24 to tubular member 12. Attachment member 26 may be circumferentially disposed about and attached to fiber optic 24 and be secured to the inner surface of tubular member 12 (e.g., distal portion 16). In at least some embodiments, attachment member 26 is proximally spaced from pressure sensor 20. Other arrangements are contemplated.

In at least some embodiments, a sealing member 28 may be disposed within tubular member 12. Sealing member 28 may be generally configured to seal or otherwise prevent body fluids that enter lumen 22 (e.g., through slots 18) from passing through tubular member 12 to more proximal regions of guidewire 10 (including the proximal end of guidewire 10 and/or outside the patient). Sealing member 28 may be positioned at a suitable location along tubular member. This may include being positioned proximal of slots 18. While a single sealing member 28 is illustrated, additional sealing members 28 may also be utilized and the additional sealing members 28 may be positioned at a suitable location along tubular member 12.

A tip member 30 may be coupled to tubular member 12. The precise form of tip member 30 can vary. For example, tip member may include a core member 32, a spring or coil 34, and a tip 36. Core member 32 may include one or more tapers. Core member 32 and/or coil 34 may be attached to tubular member 12 using a suitable attachment technique such as soldering, thermal bonding, welding, adhesive, or the like. Tip 36 may be a solder ball tip. Other tips are contemplated. In some embodiments, tip 36 may be secured directly to tubular member 12. According to these embodiments, core member 32 and/or coil 34 may be omitted from tip member 30 and/or guidewire 10.

The proximal end of guidewire 10 may be configured to attach to a connector or handle member 38. Handle 38 may include a suitable connector for a cable 40 to attached thereto and extend to another suitable device such as a signal conditioner or interferometer 42. Another cable 44 may extend from signal conditioner 42 to a suitable output device or display and/or monitoring unit 46. A clinician may utilize the readings from the display 46 to tailor the intervention to the needs of the patient or otherwise advance the goals of the intervention. These are just examples. Other devices and/or arrangements may be utilized with guidewire 10.

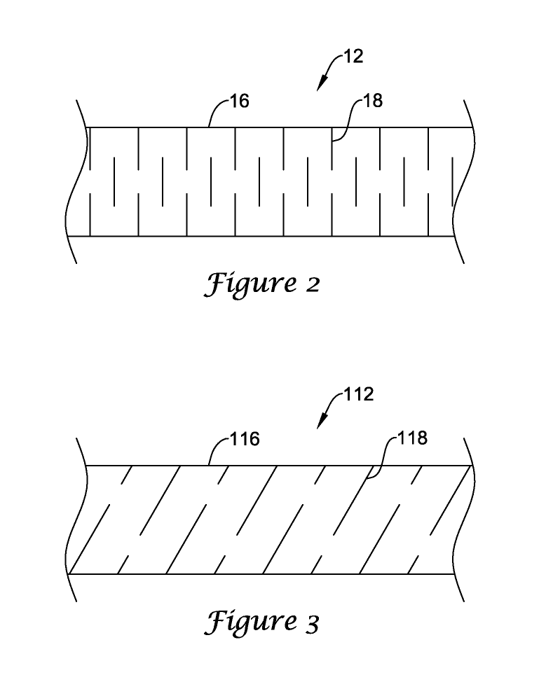

FIG. 2 illustrates distal portion 16 of tubular member 12. Here it can be seen that at least some of slots 18 may lie in a plane transverse to a longitudinal axis of tubular member 12. In this example, slots 18 are arranged in groups of opposed pairs of slots 18. Subsequent opposed pairs of slots 18 may be rotated (e.g., 90 degrees as shown or any other suitable angle). Numerous other arrangements are contemplated. For example, FIG. 3 illustrates distal portion 116 of another example tubular member 112. Here it can be seen that at least some of the slots 118 are arranged into a pattern defining an interrupted helix. These are just examples. Numerous other patterns and/or slot configurations are contemplated including those disclosed herein.

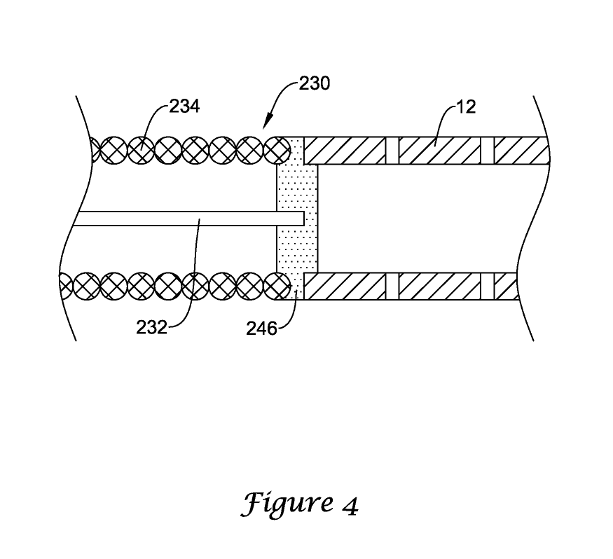

FIG. 4 illustrates a portion of another example tip member 230 that may be used with guidewire 10 (and/or other guidewires disclosed or otherwise contemplated herein). Tip member 230 may include a shaping ribbon 232 and a coil 234. Shaping ribbon 232 may be formed from a shapeable material such as, for example, stainless steel, linear elastic nitinol, other materials disclosed herein, or the like. Shaping ribbon 232 and/or coil 234 may be attached to tubular member 12 with a bonding member 246. Bonding member 246 may include an adhesive, solder, or the like, or other suitable bonding members.

The materials that can be used for the various components of guidewire 10 (and/or other guidewires disclosed herein) and the various tubular members disclosed herein may include those commonly associated with medical devices. For simplicity purposes, the following discussion makes reference to tubular member 12 and other components of guidewire 10. However, this is not intended to limit the devices and methods described herein, as the discussion may be applied to other similar tubular members and/or components of tubular members or devices disclosed herein. Tubular member 12 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of tubular member 12 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of guidewire 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into guidewire 10. For example, tubular member 12 or portions thereof may be made of a material that does not substantially distort the image and create substantial artifacts (i.e., gaps in the image). Certain ferromagnetic materials, for example, may not be suitable because they may create artifacts in an MRI image.

Tubular member 12, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of tubular member 12 that may define a generally smooth outer surface for guidewire 10. In other embodiments, however, such a sheath or covering may be absent from a portion of all of guidewire 10, such that tubular member 12 may form the outer surface. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the guidewire 10 (including, for example, the exterior surface of tubular member 12) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the sheath, or in embodiments without a sheath over portion of tubular member 12, or other portions of guidewire 10. Alternatively, the sheath may comprise a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

Various embodiments of arrangements and configurations of slots are also contemplated that may be used in addition to what is described above or may be used in alternate embodiments. For simplicity purposes, the following disclosure makes reference to guidewire 10, slots 18, and tubular member 12. However, it can be appreciated that these variations may also be utilized for other slots and/or tubular members. In some embodiments, at least some, if not all of slots 18 are disposed at the same or a similar angle with respect to the longitudinal axis of tubular member 12. As shown, slots 18 can be disposed at an angle that is perpendicular, or substantially perpendicular, and/or can be characterized as being disposed in a plane that is normal to the longitudinal axis of tubular member 12. However, in other embodiments, slots 18 can be disposed at an angle that is not perpendicular, and/or can be characterized as being disposed in a plane that is not normal to the longitudinal axis of tubular member 12.

Additionally, a group of one or more slots 18 may be disposed at different angles relative to another group of one or more slots 18. The distribution and/or configuration of slots 18 can also include, to the extent applicable, any of those disclosed in U.S. Pat. Publication No. US 2004/0181174, the entire disclosure of which is herein incorporated by reference.

Slots 18 may be provided to enhance the flexibility of tubular member 12 while still allowing for suitable torque transmission characteristics. Slots 18 may be formed such that one or more rings and/or tube segments interconnected by one or more segments and/or beams that are formed in tubular member 12, and such tube segments and beams may include portions of tubular member 12 that remain after slots 18 are formed in the body of tubular member 12. Such an interconnected structure may act to maintain a relatively high degree of torsional stiffness, while maintaining a desired level of lateral flexibility. In some embodiments, some adjacent slots 18 can be formed such that they include portions that overlap with each other about the circumference of tubular member 12. In other embodiments, some adjacent slots 18 can be disposed such that they do not necessarily overlap with each other, but are disposed in a pattern that provides the desired degree of lateral flexibility.

Additionally, slots 18 can be arranged along the length of, or about the circumference of, tubular member 12 to achieve desired properties. For example, adjacent slots 18, or groups of slots 18, can be arranged in a symmetrical pattern, such as being disposed essentially equally on opposite sides about the circumference of tubular member 12, or can be rotated by an angle relative to each other about the axis of tubular member 12. Additionally, adjacent slots 18, or groups of slots 18, may be equally spaced along the length of tubular member 12, or can be arranged in an increasing or decreasing density pattern, or can be arranged in a non-symmetric or irregular pattern. Other characteristics, such as slot size, slot shape, and/or slot angle with respect to the longitudinal axis of tubular member 12, can also be varied along the length of tubular member 12 in order to vary the flexibility or other properties. In other embodiments, moreover, it is contemplated that the portions of the tubular member, such as a proximal section, or a distal section, or the entire tubular member 12, may not include any such slots 18.

As suggested herein, slots 18 may be formed in groups of two, three, four, five, or more slots 18, which may be located at substantially the same location along the axis of tubular member 12. Alternatively, a single slot 18 may be disposed at some or all of these locations. Within the groups of slots 18, there may be included slots 18 that are equal in size (i.e., span the same circumferential distance around tubular member 12). In some of these as well as other embodiments, at least some slots 18 in a group are unequal in size (i.e., span a different circumferential distance around tubular member 12). Longitudinally adjacent groups of slots 18 may have the same or different configurations. For example, some embodiments of tubular member 12 include slots 18 that are equal in size in a first group and then unequally sized in an adjacent group. It can be appreciated that in groups that have two slots 18 that are equal in size and are symmetrically disposed around the tube circumference, the centroid of the pair of beams (i.e., the portion of tubular member 12 remaining after slots 18 are formed therein) is coincident with the central axis of tubular member 12. Conversely, in groups that have two slots 18 that are unequal in size and whose centroids are directly opposed on the tube circumference, the centroid of the pair of beams can be offset from the central axis of tubular member 12. Some embodiments of tubular member 12 include only slot groups with centroids that are coincident with the central axis of the tubular member 12, only slot groups with centroids that are offset from the central axis of tubular member 12, or slot groups with centroids that are coincident with the central axis of tubular member 12 in a first group and offset from the central axis of tubular member 12 in another group. The amount of offset may vary depending on the depth (or length) of slots 18 and can include other suitable distances.

Slots 18 can be formed by methods such as micro-machining, saw-cutting (e.g., using a diamond grit embedded semiconductor dicing blade), electron discharge machining, grinding, milling, casting, molding, chemically etching or treating, or other known methods, and the like. In some such embodiments, the structure of the tubular member 12 is formed by cutting and/or removing portions of the tube to form slots 18. Some example embodiments of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference. It should be noted that the methods for manufacturing guidewire 110 may include forming slots 18 tubular member 12 using these or other manufacturing steps.

In at least some embodiments, slots 18 may be formed in tubular member using a laser cutting process. The laser cutting process may include a suitable laser and/or laser cutting apparatus. For example, the laser cutting process may utilize a fiber laser. Utilizing processes like laser cutting may be desirable for a number of reasons. For example, laser cutting processes may allow tubular member 12 to be cut into a number of different cutting patterns in a precisely controlled manner. This may include variations in the slot width, ring width, beam height and/or width, etc. Furthermore, changes to the cutting pattern can be made without the need to replace the cutting instrument (e.g., blade). This may also allow smaller tubes (e.g., having a smaller outer diameter) to be used to form tubular member 12 without being limited by a minimum cutting blade size. Consequently, tubular member 12 may be fabricated for use in neurological devices or other devices where a relatively small size may be desired.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A pressure sensing guidewire, comprising:
a shaft having a proximal portion, a distal portion, and a distal tip portion;
wherein the distal portion has a plurality of slots formed therein; and
a pressure sensor disposed within the distal portion of the shaft.

2. The pressure sensing guidewire of claim 1, wherein the pressure sensor is an optical pressure sensor.

3. The pressure sensing guidewire of claim 1, wherein the pressure sensor is a fabry-perot pressure sensor.

4. The pressure sensing guidewire of claim 1, wherein a fiber optic cable is attached to the pressure sensor and extends proximally thererfrom.

5. The pressure sensing guidewire of claim 1, wherein the plurality of slots include at least a pair of slots lying in a plane transverse to a longitudinal axis of the shaft.

6. The pressure sensing guidewire of claim 1, wherein at least some of the plurality of slots are arranged into a pattern defining an interrupted helix.

7. The pressure sensing guidewire of claim 1, wherein the pressure sensor is attached to the distal portion of the shaft with a mount.

8. The pressure sensing guidewire of claim 1, wherein one or more sealing members are disposed within the shaft.

9. The pressure sensing guidewire of claim 1, wherein the distal tip portion includes a shaping ribbon.

10. A pressure sensing guidewire, comprising:
a tubular member having a proximal portion, a distal portion, and a lumen defined therein;
wherein the distal portion has a plurality of slots formed therein;
wherein the plurality of slots are configured to allow fluid to flow from an outer surface of the tubular member, through the slots, and into the lumen;
a pressure sensor disposed within the lumen of the tubular member and positioned within the distal portion of the tubular member; and
a tip member coupled to the distal portion of the tubular member.

11. The pressure sensing guidewire of claim 10, wherein the pressure sensor is an optical pressure sensor.

12. The pressure sensing guidewire of claim 10, wherein the pressure sensor is a fabry-perot pressure sensor.

13. The pressure sensing guidewire of claim 10, wherein a fiber optic cable is attached to the pressure sensor and extends proximally thererfrom.

14. The pressure sensing guidewire of claim 10, wherein the plurality of slots include at least a pair of slots lying in a plane transverse to a longitudinal axis of the tubular member.

15. The pressure sensing guidewire of claim 10, wherein at least some of the plurality of slots are arranged into a pattern defining an interrupted helix.

16. The pressure sensing guidewire of claim 10, wherein the pressure sensor is attached to the distal portion of the tubular member with a mount.

17. The pressure sensing guidewire of claim 10, wherein one or more sealing members are disposed within the tubular member.

18. The pressure sensing guidewire of claim 10, wherein the tip member includes a shaping ribbon.

19. A pressure sensing guidewire system, comprising:
a tubular member having a proximal portion, a slotted portion, and a lumen defined therein;
wherein the slotted portion has a plurality of slots formed therein;
wherein the plurality of slots are configured to allow fluid to flow from an outer surface of the tubular member, through the slots, and into the lumen;
an optical pressure sensor disposed within the lumen of the tubular member and positioned within the slotted portion of the tubular member;
a fiber optic cable attached to the optical pressure sensor and extending proximally therefrom;
a handle member coupled to the proximal portion of the tubular member and the fiber optic cable;
an interferometer; and
a cable extending between the handle member and the interferometer.

20. The pressure sensing guidewire system of claim 19, further comprising a display unit and a second cable extending between the interferometer and the display unit.
